(19) United States
(12) Patent Application Publication
(10) Pub. No.: US 2022/0339308 A1
(43) Pub. Date: Oct. 27, 2022

(54) LOCKABLE ENCLOSURE WITH CONTROLLED SANITIZATION CYCLE

(71) Applicant: GMI Holdings, Inc., Mt. Hope, OH (US)

(72) Inventor: Mark Soderberg, Conifer, CO (US)

(21) Appl. No.: 17/727,226

(22) Filed: Apr. 22, 2022

Related U.S. Application Data
(60) Provisional application No. 63/178,971, filed on Apr. 23, 2021.

(51) Int. Cl. A61L 2/20; A61L 2/24; A61L 2/28

(52) U.S. Cl. CPC A61L 2/202; A61L 2/24; A61L 2/28; A61L 2202/14; A61L 2202/121; A61L 2202/11

(57) ABSTRACT

A method of sanitization includes determining a door of an enclosure is closed, the door separating a compartment within the enclosure from ambient air, and performing a sanitization process. The sanitization process includes determining an article has been placed into the compartment, initiating a treatment timer, introducing a sanitization agent into a volume of the compartment with a sanitization device, and determining the treatment timer has expired. A sanitization system includes an enclosure, a controller, and a sanitization device. The enclosure is configured to receive and secure one or more articles. The enclosure has a door separating a compartment within the enclosure from ambient air. The controller is configured to determine an article has been placed into the compartment, initiate a treatment timer, instruct the sanitization device to introduce a sanitization agent into a volume of the compartment, and determine the treatment timer has expired.

### CROSS REFERENCE TO RELATED APPLICATIONS

This application claims the benefit of the filing date of and priority to U.S. Provisional Application No. 63/178,971, filed Apr. 23, 2021, entitled “Package Receptable with Decontamination System and Methods Therefore,” which is hereby incorporated by reference in its entirety.

### TECHNICAL FIELD

This disclosure is directed to fumigation, sterilization, or decontamination devices/systems. The described systems and methods may have applicability to delivery and storage enclosures for packages or goods.

### BACKGROUND

Everyday articles that people come into contact with may be susceptible to transporting germs and other microorganisms that are imperceptible to the human eye. Contact with articles contaminated with such a virus, bacteria, fungus, or other pathogen may cause infection and illness. This possibility of exposure has been known for many decades. In recent years, particularly surrounding the emergence of the coronavirus causing COVID-19, many people have become increasingly concerned about exposure to and becoming infected with a pathogen that may be capable of living outside a host organism on a surface for one or more days depending on a number of factors, including but not limited to a surface type. For example, a given pathogen may have varying survival times on hard surfaces, textiles, cardboard, and the like. Transfer of a pathogen to another individual or to a surface of an article, in some instances, may occur directly through sneezing, coughing, exhalation, etc., or may occur indirectly by touching a surface of an article after touching another surface containing the pathogen.

In order to avoid direct contact with other individuals, home delivery options have become more available in recent years, with packages, groceries, and other articles being delivered (collectively “parcels”) to consumers' homes. It is believed that a person receiving an article that has had a pathogen transferred to its surface can contact the pathogen on the article and transfer it to themselves, in some examples, by touching their own mouth, eyes, nose, or other pathways into their organ systems which may result in infection.

One recommended solution to prevent exposure and infection is to wear gloves, masks, or other personal protective equipment (PPE) when in close contact with other individuals or when interacting with articles which may potentially host a pathogen. Typically, PPE may be discarded after each use to avoid transmission of pathogens during a subsequent use. In addition to PPE, another means of safeguarding against pathogen transmission is to wipe surfaces with a disinfectant like soap, alcohol, bleach, ammonia compounds, etc. Some disinfectants may be harmful to humans and/or pets and may be difficult to completely remove from an article once used. Even soap from a soap and water mixture may be difficult to completely remove from an articular after use.

Some approaches to reducing exposure to pathogens from delivered parcels includes applying ultraviolet light, such as UV-C, from one or more lamps. However, such light is directional and may not make direct contact with certain portions of a parcel, leaving unexposed regions of the parcel susceptible to pathogen risk. Further, the irradiation level provided by such lamps may vary based on distance between the lamp and the surface being treated, which presents challenges in treating parcels of varying shapes and sizes.

A problem with current infection prevention methods is that many people do not have the discipline, desire, or knowledge to thoroughly sanitize surfaces, food, and/or other articles. As such, a need exists for improved decontamination of articles.

### SUMMARY

To improve the sanitation of a package delivery, the present disclosure contemplates a parcel delivery system utilizing a parcel receptacle in which a sanitation agent is used to sanitize the contents of the receptacle. Upon detecting that a package has been inserted, the system may activate a device that provides a sanitation agent to the receptacle. In some examples, the sanitation agent is controlled to a predetermined concentration for a predetermined amount of time. Upon completion, the sanitation agent may be purged so that a user may safely access the receptacle.

Consistent with some examples, a method of sanitization comprises determining a door of an enclosure is closed, the door separating a compartment substantially within the enclosure from ambient air. The method further comprises performing a sanitization process, the sanitization process comprising determining an article has been placed into the compartment, initiating a treatment timer, and introducing a sanitization agent into a volume of the compartment with a sanitization device. The method further comprises determining the treatment timer has expired. In some examples, the method further comprises determining the door of the enclosure has opened after initiating the treatment timer and before determining the treatment timer has expired, and reinitiating the treatment timer in response to determining the door has opened. In some examples, the method further comprises providing a visual indication of a status of the sanitization process on a display. In some examples, the method further comprises initiating a depletion timer in response to determining the treatment timer has expired, and providing an indication that the sanitization process is complete in response to expiration of the depletion timer. In some examples, the method further comprises initiating a purge timer in response to determining the treatment timer has expired, activating a purge fan configured to purge air inside the compartment to the ambient air, and deactivating the purge fan in response to determining the purge timer has expired. In some examples, the method further comprises monitoring a concentration of the sanitization agent within the compartment, wherein the treatment timer is initiated in response to the monitored concentration reaching a predetermined target concentration. In some examples, the method further comprises monitoring a concentration of the sanitization agent within the compartment, and providing an indication that the sanitization process is complete in response to determining the monitored concentration is less than a predetermined threshold. In some examples, the method further comprises locking the door of the enclosure during the sanitization process. In some examples, the method further comprises unlocking the door of the enclosure after initiating the treatment timer and before determining the treatment timer has expired in response to authenticating a received request for access into the compartment.

Consistent with some examples, a sanitization system comprises an enclosure configured to receive and secure one or more articles, the enclosure having a door separating a compartment within the enclosure from ambient air. The sanitization system further comprises a controller, and a sanitization device. The controller is configured to determine an article has been placed into the compartment, initiate a treatment timer, instruct the sanitization device to introduce a sanitization agent into a volume of the compartment, and determine the treatment timer has expired. In some examples, the system further comprises a locking mechanism configured to receive a command from the controller and actuate a lock in response to the received command, wherein the controller is further configured to transmit a command to the locking mechanism to actuate the lock to a locked configuration prior to introducing the sanitization agent into the volume. In some examples, the controller initiates a depletion timer in response to determining the treatment timer has expired, and prevent unlocking of the locking mechanism until expiration of the depletion timer. In some examples, the system further comprises one or more sensors, wherein the controller is configured to monitor a concentration of the sanitization agent within the volume based on signals received from the one or more sensors. In some examples, the system further comprises one or more purge fans in operative communication with the controller, wherein the controller is further configured to activate the one or more purge fans, monitor the concentration of the sanitization agent within the volume, and deactivate the one or more purge fans in response to determining the concentration is below a threshold value. In some examples, the controller determines the monitored concentration of the sanitization agent within the volume is less than a target concentration, and instruct the sanitization device to dispense an additional quantity of sanitization agent in response to determining the monitored concentration is less than the target concentration. In some examples, the controller delays the initiation of the treatment timer or modify a timer value of the treatment value based on the monitored concentration of the sanitization agent within the volume.

Consistent with some examples, a parcel receptacle comprises an enclosure configured to receive and secure one or more parcels, the enclosure having a door separating a compartment within the enclosure from ambient air. The parcel receptacle further comprises a sanitization device configured to introduce a sanitization agent into a volume of the compartment, and a controller. The controller is configured to communicate with a remote access device to selectively provide access into the compartment to delivery personnel, determine a parcel has been placed into the compartment, initiate a treatment timer, instruct the sanitization device to introduce the sanitization agent into the volume of the compartment, determine the treatment timer has expired, and provide a visual indication that the treatment timer has expired. In some examples, the sanitization device is an ozone generator and the sanitization agent is ozone. In some examples, the controller initiates a depletion timer in response to expiration of the treatment timer, and prevents access into the compartment until expiration of the depletion timer, wherein a timer value of the depletion timer is based on a period of time associated with degradation of ozone. In some examples, the system further comprises one or more ozone sensors, wherein the controller is configured to monitor a concentration of ozone within the volume based on signals received from the one or more sensors, and one or more purge fans in operative communication with the controller. In some examples the controller is configured to activate the one or more purge fans, monitor the concentration of ozone within the volume during operation of the one or more purge fans, and deactivate the one or more purge fans in response to determining the monitored concentration is below a threshold value.

It is to be understood that both the foregoing general description and the following drawings and detailed description are exemplary and explanatory in nature and are intended to provide an understanding of the present disclosure without limiting the scope of the present disclosure. In that regard, additional aspects, features, and advantages of the present disclosure will be apparent to one skilled in the art from the following detailed description and the drawings.

### BRIEF DESCRIPTION OF THE DRAWINGS

The accompanying drawings illustrate implementations of the systems, devices, and methods disclosed herein and together with the description, serve to explain the principles of the present disclosure.

FIG. 1 illustrates examples of enclosures that may benefit from the sanitization and disinfection systems and methods described herein.

FIG. 2a illustrates an example decontamination system.

FIG. 2b is a schematic illustration of an example parcel receptacle in accordance with the present disclosure.

FIG. 2c illustrates an example of a parcel delivery system in accordance with the present disclosure.

FIGS. 3-6 each illustrate a flow chart of examples of a method of disinfecting an article.

These Figures will be better understood by reference to the following Detailed Description.

### DETAILED DESCRIPTION

Some examples described herein automate a sanitizing process for various articles. As used herein, it should be appreciated that the terms “disinfect,” “decontaminate,” “sanitize,” “sterilize,” “cleanse,” “fumigate,” and variations thereof may be used interchangeably and generally refer to reducing a population of, inhibiting, or otherwise weakening a pathogen, such as but not limited to a virus, bacteria, fungus, or any other unwanted contaminant. It should be appreciated that the concepts described herein may be modified, combined, or elements thereof rearranged in any suitable manner without departing from the scope of the present disclosure.

In some examples, systems and methods may sanitize an article by introducing a liquid, gas, vapor, or other suitable agent for sanitization (collectively, “sanitization agents”) which is circulated inside a closed and most often locked enclosure, such as a package locker. For purposes of this disclosure, the term “vapor” may refer to any sprayed, vaporized, or aerosolized liquid or solid material suitable for use in the systems and methods of the present disclosure. An example of a gas that may be used as a sanitization agent includes ozone (O₃), which may be effective on various pathogens including coronaviruses. It has been published by the U.S. Food and Drug Administration (FDA) that ozone is a safe and effective treatment to help eliminate pathogens on exposed surfaces. Although examples described herein recite the use of ozone, other sanitization agents (which are now known or may be identified in the future) may be employed without departing from the scope of the present disclosure. By using a forced circulation of a sanitization agent within an enclosure, the agent may be able to reach and/or contact essentially all exposed surfaces of article which may be susceptible to contaminants, such as packages or groceries.

As described in some examples below, an enclosure may be locked and a gas may be circulated throughout an internal compartment of the enclosure housing articles placed into the enclosure. A concentration of the gas may be monitored and controlled, as may a time of application of the gas. The enclosure may remain locked until treatment is complete.

Examples described herein may pertain to sanitization of the surfaces of articles that have been received by an individual, a family, a business, by a delivery person for subsequent delivery, or by employees during the handling of raw materials or products in manufacture. In some examples, articles are packages containing products ordered online, mail, groceries, meal delivery kits, or other items delivered to or from one person or company to another.

The systems and methods described herein may be beneficial in combating the spread of illnesses, particularly during pandemics such as the recent coronavirus pandemic given the associated increase in online purchasing and home delivery. Although efforts have been made by individuals and companies to identify and implement precautions to reduce the spread of pathogens, some measures may not completely eradicate pathogens and additional measures in the chain of transmission of articles from vendors to delivery services and ultimately to consumers may be beneficial. While some recommendations made during the recent coronavirus pandemic suggested that contact with delivered articles should be avoided for 24 to 48 hours after delivery to deprive pathogens of a host required for survival and replication. Such recommendations are often impractical, for example, in the instance of a grocery delivery with food items or a pharmaceutical delivery with a medication requiring proper storage conditions.

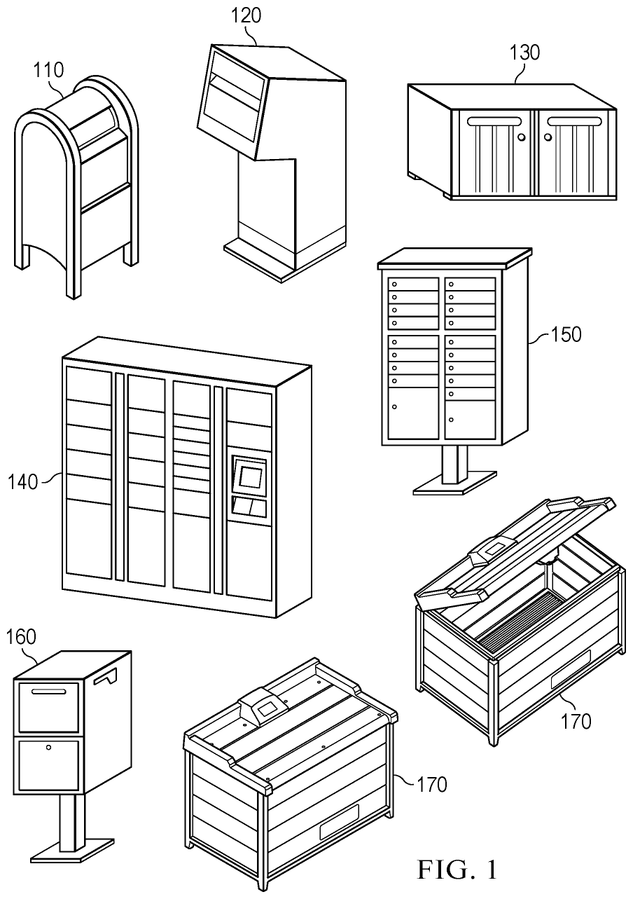

FIG. 1 illustrates examples of enclosures that may be suitable for implementation of the sanitization systems and methods described herein. These various examples may be referred to as “controlled space containers” or “enclosures” in that they are receptacles for various articles and provide restricted access to their contents. Examples herein describe sanitization systems that may be used with the described enclosures, however it should be understood that systems and methods for sanitization described herein may be suitable for use in other types of enclosures and in other applications with confined volumes. Additionally, the enclosures or features of the enclosures may be considered components of one or more examples of a sanitization system as described herein.

The systems and methods for sanitization of articles described herein are contemplated for use in a wide array of enclosures. In some examples, a sanitization system or method as described herein may be applicable to a mailbox 110, such as a USPS mail receptacle to reduce pathogen exposure to USPS personnel from deposited envelopes and packages or other parcels. In some examples, a sanitization system or method as described herein may be applicable to a package receptacle 120, such as a UPS or FedEx drop box, to reduce pathogen exposure to carrier employees from deposited parcels. In some examples, a sanitization system or method as described herein may be applicable to a lock box 130, such as a keyed or combination locked box used by medical facilities, commercial buildings, professional offices, etc. to send and/or receive parcels, lab samples, and other articles, to reduce pathogen exposure to employees of such facilities as well as vendors with access to the lock box. In some examples, a sanitization system or method as described herein may be applicable to a deliver kiosk 140, such as an interactive package pickup system, to reduce pathogen exposure to package recipients retrieving ordered articles from secure compartments or lockers using a user interface and to carriers retrieving returned articles from such lockers. In some examples, a sanitization system or method as described herein may be applicable to a community mailbox 150, such as a group mailbox that condominiums and neighborhoods often employ, to reduce pathogen exposure to individuals within the community. In some examples, a sanitization system or method as described herein may be applicable to a home mailbox, such as a typical residential mailbox or drop box, to reduce pathogen exposure to homeowners or residents. In some examples, a sanitization system or method as described herein may be applicable to a delivery receptacle, such as a locking package receptacle 170 (illustrated in FIG. 1 with a lid in both a closed and open position) or vault that is designed for preventing package theft to residential or commercial buildings, to reduce pathogen exposure to delivery recipients from deposited parcels, groceries, or other articles as well as to reduce pathogen exposure to delivery drivers retrieving outbound parcels from the locking package receptacle 170. The delivery receptacle 170 may provide controlled access (e.g., using an electronically-actuated lock) and/or an interactive display that may be utilized to perform or initiate various functions described herein. In some examples, a sanitization system or method as described herein may be provided via a retrofit kit for installation into an existing enclosure.

While there are a variety of sanitization agents that can potentially be used for sanitization, there are a number of factors that must be evaluated in determining their suitability for use in various enclosures. For example, some agents may have a toxicity that either precludes their use or that warrants appropriate management to reduce or prevent undesirable exposure to the agent itself. Some agents have particular temporal considerations regarding efficacy related to exposure-time. Some agents may be cost prohibitive or impractical given the expense associated with them. Some agents may be difficult to obtain due to their market availability, material handling license requirements, etc. While the present disclosure contemplates the use of any suitable sanitization agent including, but not limited to chlorine dioxide, glutaraldehyde, paraformaldehyde, ethylene oxide, peracetic acid, or hydrogen peroxide, practical considerations may render some agents more suitable than others. Some may have toxicity implications preventing their use altogether while others may have toxicity implications than can be overcome through appropriate management. In some examples, a sanitization agent which may otherwise present concerns regarding toxicity may be rendered nontoxic by exposing the agent to an appropriate catalyst before, during, or after use for sanitization of articles.

In some examples, an exchangeable cartridge or canister may contain a sanitization agent for use in the systems and methods of the present disclosure. Such cartridges may be replaced with a full cartridge when the previous cartridge is empty to replenish a sanitization system when needed. In some examples, a storage tank of (or in fluid communication with) a sanitization system may be refillable with a suitable sanitization agent.

In some examples, a sanitization agent used in a sanitization system or method described herein may be ozone. The United States public health agency Centers for Disease Control and Prevention has published on their website: “Ozone has been used for years as a drinking water disinfectant. Ozone is produced when O₂ is energized and split into two monatomic (O₁) molecules. The monatomic oxygen molecules then collide with O₂ molecules to form ozone, which is O₃. Thus, ozone consists of O₂ with a loosely bonded third oxygen atom that is readily available to attach to, and oxidize, other molecules. This additional oxygen atom makes ozone a powerful oxidant that destroys microorganisms but is highly unstable (i.e., half-life of 22 minutes at room temperature).”

The instability of ozone may be advantageous for utilizing ozone in sanitization because the gas may break down rapidly and be naturally rendered harmless. Ozone generators are available as small and relatively inexpensive devices that operate by passing air (including O₂) through an electric field. Ozone generators allow ozone to be locally generated from air in the environment without replenishment of cartridges or other storage containers. The generated ozone may be effective for sanitizing a wide variety of articles. Ozone generators may be ideal for delivery receptacles as such enclosures are relatively small with compartments having defined volumes in which the atmosphere can be readily managed.

An example of a method of sanitizing one or more articles in an enclosure may include a treatment cycle in which a controller determines that one or more articles have been placed in the enclosure using one or more sensors. Ozone may be introduced into the compartment of the enclosure to treat the articles with a target concentration (e.g., ppm) of ozone for a prescribed period of time. The space may then be purged with air from the surrounding environment or a waiting period may be implemented to permit the ozone to degrade naturally to oxygen (O₂) until an innocuous concentration is achieved in the compartment of the enclosure. A notification may be provided to a user indicating that the treatment cycle is complete. In some examples, the target concentration may be in the range of about 3 to about 5 parts per million (ppm) or more and the innocuous concentration may be about 0.3 ppm or less.

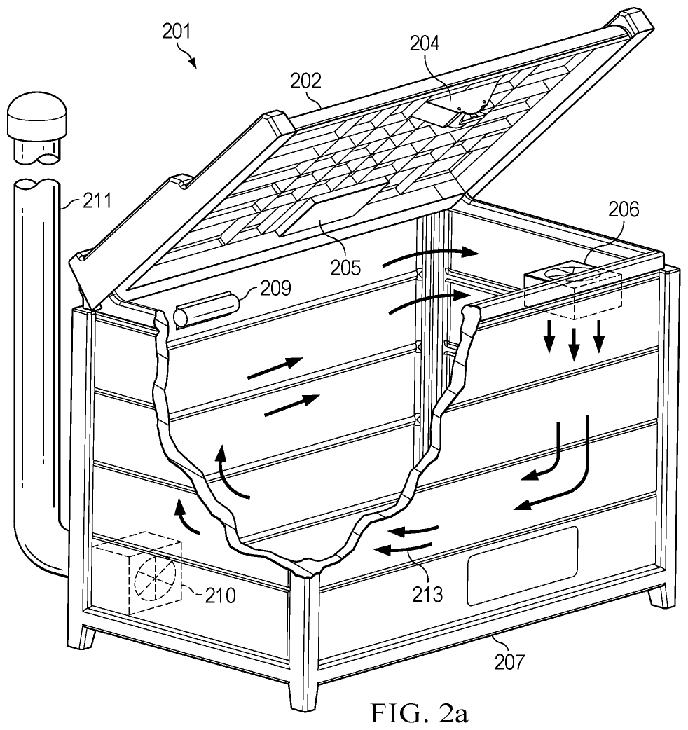

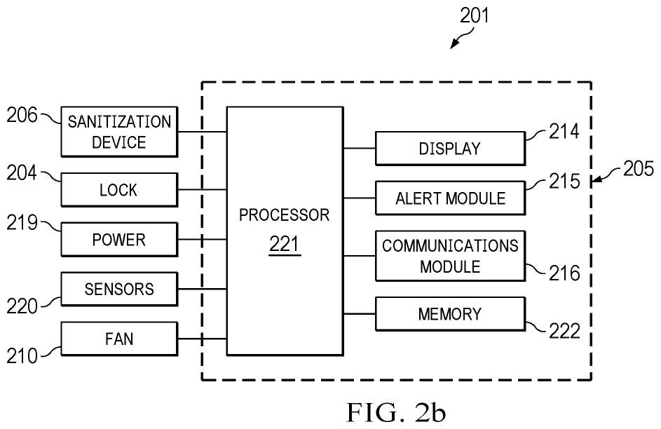

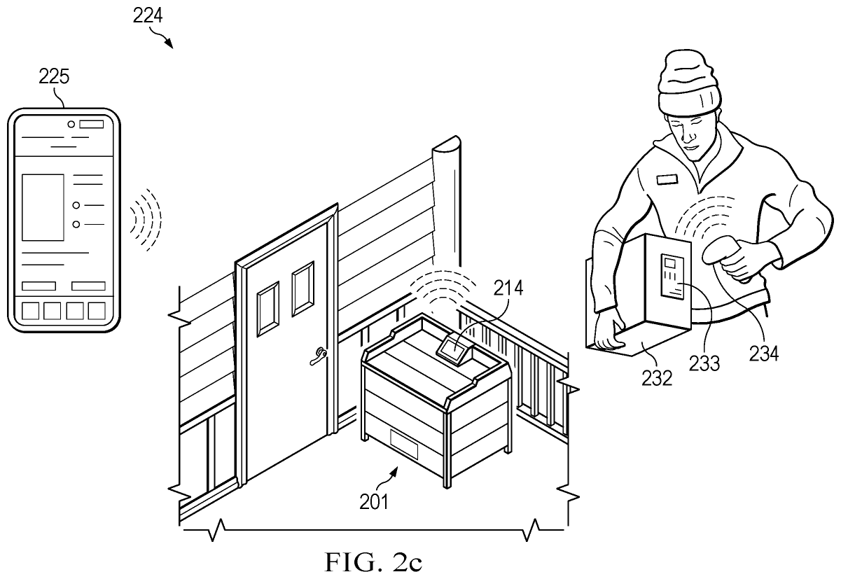

FIG. 2a illustrates an example of a system for sanitization treatment of articles that includes a sanitization device, such as an ozone generator. The system includes a delivery receptacle 201 (which may be similar to delivery receptacle 170 of FIG. 1). The delivery receptacle 201 includes a door or lid 202 (used interchangeably herein) and a compartment 207 with a floor, walls, and the lid 202 defining a volume of the compartment. In this example, an electronic lock 204 can be engaged to latch the lid 202 closed and provided selective access into the compartment. Electronic lock 204, or a separate sensor, may provide feedback to controller 205 indicating whether lid 202 is opened or closed and whether the delivery receptacle is locked (e.g., lock mechanism 204 is engaged) or unlocked. The controller 205 may include one or more processors, memory devices, and communications modules (e.g., Wi-Fi radio), and can be used to control sanitization of articles within the compartment and/or the surfaces of the compartment itself. The delivery receptacle 201 includes a display 214 having a user interface (“UP”) (shown in FIG. 2c). The UI may include an LED or LCD panel, indicator lights, speakers, push buttons, a touchscreen, or the like. Functions of the delivery receptacle 201 including access control and sanitization may be initiated via user interaction with the UI and/or via wireless connection with controller 205 using a remote interface (e.g., on a wireless device such as a smartphone).

The delivery receptacle 201 includes one or more sanitization devices 206 for providing a sanitization agent, such as an ozone generator or other sanitization agent dispenser (e.g., a vaporizer). A sanitization device 206 may include a regulator for controlling a flow rate, volume, concentration, and/or other parameter of a sanitization agent. The sanitization device 206 may also include a fill-level sensor for monitoring an available volume of a sanitization agent. In some examples, the fill-level sensor may include one or more of an ultrasonic sensor, a capacitive sensor, a float, an optic sensor, camera, or any other point level or continuous level sensing devices.

A sanitization device 206 may include an integrated circulation fan which will affect circulation of a sanitization agent throughout the compartment 207. In examples in which the sanitization device includes an ozone generator, the integrated circulation fan and/or an accessory fan placed within the compartment may be orientated in any suitable direction to achieve a desired circulation pattern. An example circulation pattern is depicted in FIG. 2a as arrows 213. In some examples, the sanitization device 206 is mounted near the lid 202 of the delivery receptacle 201. Because ozone is heavier than air, it will tend to settle toward the floor of the compartment and the circulation fan(s) may help lift the ozone away from the floor. The circulation pattern may be configured based upon the shape and size of the compartment 207 and/or based upon the dimensions received articles to optimize sanitization agent exposure across surfaces of received articles.

In some examples, an agent sensor 209 may be disposed within the compartment. The agent sensor 209 may be controlled by and/or may transmit signals to the controller 205 and/or sanitization device 206. The agent sensor 209 may be used for concentration monitoring and/or to provide feedback for dispensing of a sanitization agent. For example, the agent sensor 209 may be used to control the rate of ozone production within the compartment 207 and to monitor depletion of ozone during purging.

A purge fan 210 is disposed in the compartment 207 to facilitate active purging of a sanitization agent after a treatment cycle to more quickly reduce sanitization agent concentration. Again, because ozone and some other sanitization agents are heavier that air, the purge fan 210 may be disposed near the floor of the compartment 207. The purge fan 210 may be off during a treatment cycle and may be activated upon completion of the cycle. The purge fan 210 may also include or be positioned adjacent to a valve or door that is actuated by the controller to be closed during treatment and opened in conjunction with activation of the purge fan. Depending on physical constraints such as a location of the delivery receptacle 201, an exhaust vent 211 may be provided to discharge the sanitization agent to a preferred location. The exhaust vent 211 may be a short section of pipe extending a short distance (e.g., a few feet) from the delivery receptacle 201 or may include a substantial length of piping to deposit the sanitization agent at a location remote from the delivery receptacle 201.

It should be appreciated that delivery receptacles and other enclosures (such as those illustrated in FIG. 1) are commonly located outdoors where the sanitization agent can be readily diluted into ambient air. In indoor applications, the exhaust vent 211 may extend to an outdoor location in a similar manner to a clothes dryer vent.

The agent sensor 209 may be monitored to determine when the sanitization agent has reached an innocuous concentration. During treatment and prior to reaching an innocuous concentration, the controller 205 may not permit unlocking and opening of the lid 202 to prevent exposure of users to high concentrations of the sanitization agent. A request to unlock the lid 202 received at the UI of the display 214 or via a remote interface may be ignored or discarded while the sanitization agent remains above the innocuous concentration. A notification may be provided to user requesting to open the lid 202 that a treatment is in progress and/or that access is not currently permitted. An estimated time remaining in the treatment cycle or waiting period may also be provided to a user, via the display 214 or wireless communication to a remote interface. Upon reaching an innocuous concentration, the controller may permit the package receptacle 201 to be unlocked and opened. A notification may be provided to a user indicating that the package receptacle 201 is ready to provide access. In some examples, the agent sensor 209 may monitor the concentration of the sanitization agent during a waiting period following treatment while the purge fan 210 is off. Upon reaching a defined concentration, the controller 205 may activate the purge fan 210 to purge the compartment 207. This may help ensure that concentrations of the sanitization agent in exhausted air from the purging process are sufficiently low. The defined concentration for activation of the purge fan 210 may equal to the innocuous concentration or may be between the innocuous concentration and the target concentration as the purging process will typically blend the purged air with ambient air, rapidly lowering the concentration level.

It may be desirable to provide a plurality of treatment cycles of varying degrees and with various optional functions. In some examples, a plurality of available treatment cycles may be selectable by a user. For example, a basic or standard cycle may be provided for typical everyday use, a rapid cycle may be provided for urgent sanitization, and/or a comprehensive elevated cycle may be provided for high-risk articles. In some examples, the target concentration for use in a medium-duty cycle may be higher than the target concentration of a light-duty cycle. Similarly, the target concentration for use in a heavy-duty cycle may be higher than the target concentration of a medium-duty cycle. In some examples, the target concentration may be the same across all available treatment cycles. In some examples, the treatment timer value for a medium-duty cycle may be higher than the treatment timer value for a light-duty cycle and/or the treatment time value for a heavy-duty cycle may be higher than the treatment timer value for a medium-duty cycle. The treatment timer value may be the same across all available treatment cycles, for example, based on a worst case condition requiring a maximum length of time. Any number of additional cycles with varying parameters may be provided as available options and selected as desired. In some examples, a user may be able to configure individual parameters associated with a treatment cycle using, for example, the user interface of the display 214 or through a wireless connection between the controller 205 and a remote interface. User configurable parameters may include a length of treatment time, a target concentration of sanitization agent, a selection of one or more sanitization agents from a plurality of available sanitization agents, whether to use active purging at completion of the treatment, etc. In some examples, parameters for sanitization may be automatically determined by the controller 205 based on a number of factors including, but not limited to, a type of article received (e.g., parcel or grocery), sensor measurements, and/or prior use history.

FIG. 2b schematically illustrates components of an example of a parcel receptacle 201. The parcel receptacle 201 may include a controller 205, a lock mechanism 204, a power source 219, and one or more sensors 220. The controller 205 of the parcel receptacle 201 includes a display 214, a processor 221, memory 222, an alert module 215, and a communications module 216. The processor 221 is in operative communication with each of the other components of the controller 205 and is also in operative communication with other components of the parcel receptacle 201 outside the controller 205 such as the lock mechanism 204, the power source 219, and one or more sensors 220. The display 214 may display information to a user such as a current status of the parcel receptacle 201 (e.g., locked/unlocked, sanitizing status, etc.), display instructions to a driver (e.g., “Enter Access Code”), and/or may receive user inputs (e.g., an access code or a menu item selection). Memory 222 may store instructions for operation of the various functions of the parcel receptacle 201 by the controller 205.

Memory 222 may also store parameters associated with a sanitation process. For example, target ozone concentrations, sanitation times, etc. For example, a “sanitation time” may be maintained by the processor 221 in memory 222 which is either pre-programmed to a fixed value, or is user-settable via the user interface, or by receiving a message via the communications module 216.

The alert module 215 may include a speaker and/or a light that can be activated to alert a user of one or more conditions. For example, a light may indicate that the sanitation agent concentration is above a threshold, or that the receptacle 201 is currently performing a sanitation procedure. In some examples, the alert module 215 may be configured to perform functions of the display 214 described herein. For example, indicating the status of a sanitization cycle, status of the lock, etc. In some examples, the alert module 215 is incorporated into the display 214, rather than being a separate component.

The communications module 216 may include one or more transmitters, antennas, transceivers, receivers, etc. to facilitate wireless communication with external devices including, but not limited to, a mobile device (e.g., mobile device 225 of FIG. 2c), a customer's router, or a driver device (e.g., driver device 234 of FIG. 2c). The communications module 216 may be configured for communication using one or more of a Wi-Fi protocol, a cellular protocol, a Bluetooth protocol, etc.

The one or more sensors 220 may include a number of different types of sensors configured to transmit signals to the processor 221. In some examples, sensors 220 may include one or more sensors configured to detect the presence of a parcel. Further, sensors 220 may include sensors that are not directly related to detecting the presence of a parcel. For example, a lock mechanism actuation sensor may be configured to monitor a portion of a lock mechanism 204 such that the controller 205 can determine whether the lock mechanism is in a locked configuration or a closed configuration. Similarly, sensors 220 may include one or more sensors configured to monitor a position of the lid to determine whether the lid is open or closed. Sensors 220 may also include one or more agent sensors (e.g., agent sensor 209) for use in monitoring and control of a sanitization process.

It should be appreciated that FIG. 2b illustrates one example of a parcel receptacle 201. It is contemplated that additional components may be included and some of the enumerated components may be omitted without departing from the scope of this disclosure.

FIG. 2c illustrates a parcel delivery system 224 including parcel receptacle 201, a driver device 234, and a mobile device 225. The various devices may communicate via a plurality of local and remote data communication channels. Locally, a user may be able to control the parcel receptacle 201 (e.g., access permissions and control a sanitation process) via the mobile device 225 and a driver may be able to access the parcel receptacle 201 using the driver device 234. Zebra Technologies Corp. markets devices which may be suitable for use as a driver device 234. Each of the parcel receptacle 201, the driver device 234, and the mobile device 225 may be configured to communicate with remote processing platforms via the internet and may be configured to communicate with each other via direct local communications channels, a local network, or through the internet.

The parcel receptacle 201 may be disposed at any suitable commercial or residential location. In the illustrated example, the parcel receptacle is positioned on a front porch of a home. A user may be able to run an application on the mobile device 225 to monitor and control the parcel receptacle 201. For example, a user may be able to add or remove permissions for other users, including carriers and drivers, may be able to lock or unlock the parcel receptacle 201 from a user interface, may control a sanitation process, receive a notification that a sanitation process has been completed, etc. The mobile device 225 may communicate with the parcel receptacle 201 via a local data communication channel when within range and may communicate with the parcel receptacle 201 indirectly via the interne when out of range, typically using the customer's Wi-Fi router. In this regard, a user may be able to control the parcel receptacle 201 via the mobile device 225 even when located remotely from the premises.

A driver may have a hand-held delivery driver device 234 that may be configured to scan a label 233 on a parcel 232 to extract or lookup a parcel identifier such as a carrier tracking number. The driver device 234 may utilize a local data communication channel to communicate a request to unlock the parcel receptacle 201 to allow the driver to put the parcel in the receptacle 201.

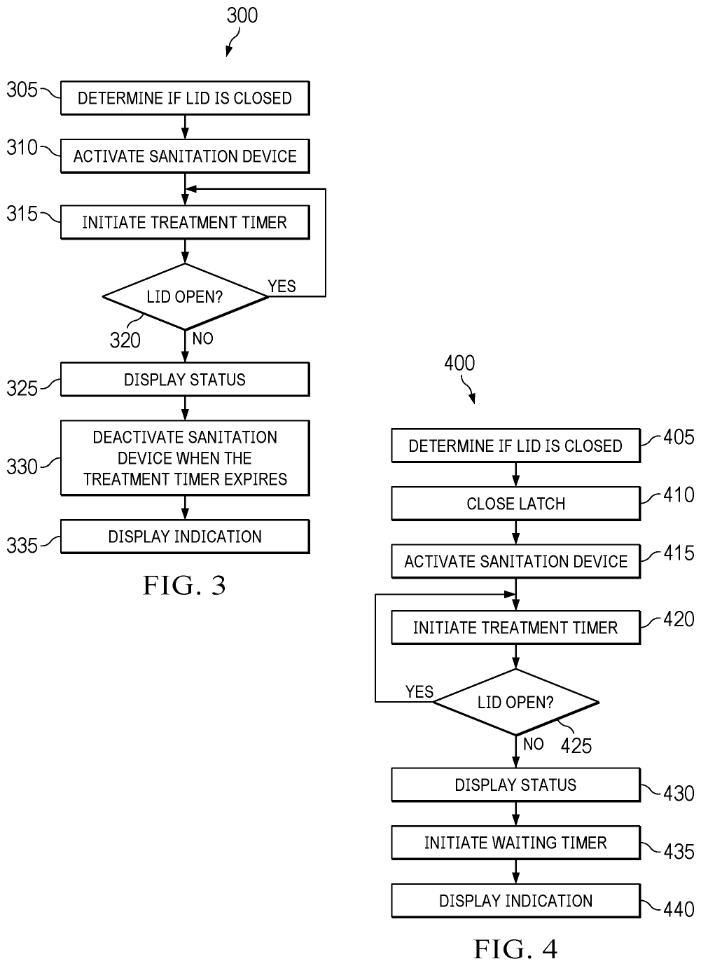

FIGS. 3-6 described below illustrate examples of methods utilizing treatment cycles having various functions or processes. The methods are described with reference to the system for sanitization illustrated in FIGS. 2a, 2b, and 2c and described above. However, it should be appreciated that these methods are suitable for use in a variety of enclosures including, but not limited to, any of those illustrated in FIG. 1. Certain processes of these methods are described with reference to components that may be specific to the system of FIGS. 2a, 2b, and 2c, but it is contemplated that processes specific to any particular example system may be omitted, for example when implementing the methods with respect to other types of enclosures which do not include such components. Each of these methods may be initiated automatically by a controller upon detecting receipt of an article, periodically at defined intervals, or otherwise. These methods may alternatively or additionally be initiated by a user through interaction with a user interface at the enclosure or on a remote interface.

FIG. 3 depicts an example of a method of sanitization 300. At process 305, a controller (e.g., controller 205) associated with an enclosure (e.g., delivery receptacle 201) determines whether a lid or door (e.g., lid 202) of the enclosure is currently open or closed. Closing of the lid may be signaled to the controller by a sensor (e.g., a sensor monitoring the electronic lock 204) upon closing, the controller may send an inquiry to the sensor prompting a return signal indicating the current status, or the controller may maintain a status of a lock based on previously sent instructions to control the lock. The controller may also determine whether an article has been received in the compartment. In this regard, the controller may: 1) assume that an article has been placed in the compartment as a result of the lid having been closed by a user; 2) assume that an article has been placed in the compartment as a result of having received and/or authenticated a request for access from a driver device; and/or 3) a sensor may indicate to the controller that the presence of a package has been detected. The latter may help distinguish from a closing of the lid after removal of a package from the enclosure. Further, any other suitable techniques may be used to determine that an article has been placed in the compartment.

At process 310, one or more sanitization devices (e.g., sanitization device 206), which may include an ozone generator, are activated. A sanitization device may be configured to dispense a sanitization agent (e.g., generate ozone) at a maximum output capacity, or the dispensing may be regulated, for example by a proportional-integral-derivative (PID) control loop, by a controller tasked with management of the treatment cycle (which may be the controller of the enclosure or a controller of the sanitization device), or by a flow regulator. The sanitization device may have a fan which can be activated to help circulate the sanitization agent throughout the compartment. Additional accessory fans in disposed in locations within the compartment may also be activated to aid in agent circulation. One or more sensors may monitor a concentration level of a sanitization agent within the compartment or monitor a dispensed volume of the sanitization agent as it flows from the sanitization device. The one or more sensors may send monitoring signals to the controller managing the treatment cycle.

At process 315, the controller tasked with management of the treatment cycle initiates a treatment timer for executing a defined period of exposure of the article to the sanitization agent. The defined period may be determined based on a variety of factors including, but not limited to, a type of article received, a type of sanitization agent being used, a concentration of the sanitization agent, a type of pathogen expected, etc. For example, a 30-minute treatment period may be a known effective amount of time for an ozone concentration of approximately 3 ppm to effectively sanitize a cardboard box.

At process 320, the controller determines whether the lid has been opened. If so, the method may return to process 315, restarting the treatment timer. This interruption of the method may occur, for example, if a subsequent delivery is made following the initiation of the treatment cycle. If it is determined that the lid has not been opened, the method continues to process 325. In some examples, if the method must return to process 315, the controller may determine a reduced treatment timer value is sufficient based on a period of exposure that is determined to have occurred before the interruption in the treatment cycle. This determination may be based in part on the concentration of sanitization agent previously dispensed, the period of exposure the article received prior to the lid being opened, the duration of the lid being open, and/or the concentration of sanitization agent remaining after closing of the lid. In some examples, the controller may determine that it is necessary to return to process 310 and dispense additional sanitization agent if the concentration was reduced by more than an acceptable amount by the lid being opened.

At process 325, a user interface on the display 214 indicates the status of the treatment cycle. For example, a certain color of light may be activated indicating a stage of the treatment cycle in process. A display which is part of user interface may indicate the treatment cycle is ongoing and may indicate a remaining time left in the cycle. Audio cues may also be provided by the user interface. The controller may also transmit a status of the treatment cycle to a remote interface, for example, via a wireless communication module.

At process 330, when the treatment timer expires, the sanitization device is deactivated. A timer associated with a waiting period may be initiated (“depletion timer”). The waiting period timer value may be user selected via the user interface or may be controlled by wireless connection to controller. The controller may also determine the waiting period timer value based on any number of factors including, but not limited to, sensor data, the volume of the compartment, a type of article delivered, a type of sanitization agent used, and/or the dispensing capacity of the sanitization device. In some examples of method 300, the waiting period timer may be based on the time it takes for a sanitization agent to naturally break down and/or escape the compartment and to achieve an innocuous concentration inside the compartment. Upon expiration of the waiting period timer, the electronic lock may automatically unlock or may be permitted to be unlocked by a user.

At process 335, the user interface of the enclosure may provide an indication to a user that the treatment cycle has finished and the lid may be opened for removal of the article. For example, a particular light may be activated or a message displayed on the display screen. A notification may also be sent to a user's mobile device or other remote interface.

FIG. 4 depicts another example of a method of sanitization 400. Method 400 shares similar elements with method 300 and may include addition of further processes performed by the system.

At process 405, a controller (e.g., controller 205) associated with an enclosure (e.g., delivery receptacle 201) determines whether a lid or door (e.g., lid 202) of the enclosure is currently open or closed. Closing of the lid may be signaled to the controller by a sensor (e.g., a sensor monitoring the electronic lock 204) upon closing, the controller may send an inquiry to the sensor prompting a return signal indicating the current status, or the controller may maintain a status of a lock based on previously sent instructions to control the lock. The controller may also determine whether an article has been received in the compartment. In this regard, the controller may: 1) assume that an article has been placed in the compartment as a result of the lid having been closed by a user; 2) assume that an article has been placed in the compartment as a result of having received and/or authenticated a request for access from a driver device; and/or 3) a sensor may indicate to the controller that the presence of a package has been detected. The latter may help distinguish from a closing of the lid after removal of a package from the enclosure. Further, any other suitable techniques may be used to determine that an article has been placed in the compartment.

At process 410, an electronic lock (e.g., electronic lock 204) latches to secure the lid of the enclosure. The locking may be a required feature of the sanitization system or may be an option which is enabled by the user via a local or remote user interface. In some examples, the enclosure may allow temporary access to a delivery person to enable placement of additional articles in the enclosure. This may be permitted by receipt of an appropriate security code via a local or remote user interface or by authentication processes executed between the controller and a remote access device (e.g., a package scanner or other delivery driver device).

At process 415, one or more sanitization devices (e.g., sanitization device 206), which may in some examples include an ozone generator, are activated. A sanitization device may be configured to dispense a sanitization agent (e.g., generate ozone) at a maximum output capacity, or the dispensing may be regulated, for example by a proportional-integral-derivative (PID) control loop or by cycling the sanitization device off when a desired threshold is reached and back on when the concentration falls below or nears the threshold within a predefined or programmable amount, by a controller tasked with management of the treatment cycle (which may be the controller of the enclosure or a controller of the sanitization device), or by a flow regulator. The sanitization device may have a fan which can be activated to help circulate the sanitization agent throughout the compartment. Additional accessory fans in disposed in locations within the compartment may also be activated to aid in agent circulation. One or more sensors may monitor a concentration level of a sanitization agent within the compartment or monitor a dispensed volume of the sanitization agent as it flows from the sanitization device. The one or more sensors may send monitoring signals to the controller managing the treatment cycle.

At process 420, the controller tasked with management of the treatment cycle initiates a treatment timer for executing a defined period of exposure of the article to the sanitization agent. The defined period may be determined based on a variety of factors including, but not limited to, a type of article received, a type of sanitization agent being used, a concentration of the sanitization agent, a type of pathogen expected, etc. For example, a 30-minute treatment period may be a known effective amount of time for an ozone concentration of approximately 3 ppm to effectively sanitize a cardboard box.

At process 425, the controller determines whether the lid has been opened. If so, the method may return to process 420, restarting the treatment timer. This interruption of the method may occur, for example, if a subsequent delivery is made following the initiation of the treatment cycle. If it is determined that the lid has not been opened, the method continues to process 430. In some examples, if the method must return to process 420, the controller may determine a reduced treatment timer value is sufficient based on a period of exposure that is determined to have occurred before the interruption in the treatment cycle. This determination may be based in part on the concentration of sanitization agent previously dispensed, the period of exposure the article received prior to the lid being opened, the duration of the lid being open, and/or the concentration of sanitization agent remaining after closing of the lid. In some examples, the controller may determine that it is necessary to return to process 415 and dispense additional sanitization agent if the concentration was reduced by more than an acceptable amount by the lid being opened.

At process 430, a user interface of the display 214 indicates the status of the treatment cycle. For example, a certain color of light may be activated indicating a stage of the treatment cycle in process. A display which is part of user interface may indicate the treatment cycle is ongoing and may indicate a remaining time left in the cycle. Audio cues may also be provided by the user interface. The controller may also transmit a status of the treatment cycle to a remote interface, for example, via a wireless communication module.

At process 435, a timer associated with a waiting period may be initiated. The waiting period timer value may be user selected via the user interface or may be controlled by wireless connection to controller. The controller may also determine the waiting period timer value based on any number of factors including, but not limited to, sensor data, the volume of the compartment, a type of article delivered, a type of sanitization agent used, and/or the dispensing capacity of the sanitization device. In some examples of method 400, the waiting period timer may be based on the time it takes for a sanitization agent to naturally break down and/or escape the compartment and to achieve an innocuous concentration inside the compartment. Upon expiration of the waiting period timer, the electronic lock may automatically unlock or may be permitted to be unlocked by a user.

At process 440, the user interface of the enclosure may provide an indication to a user that the treatment cycle has finished and the lid may be opened for removal of the article. For example, a particular light may be activated or a message displayed on the display screen. A notification may also be sent to a user's mobile device or other remote interface.

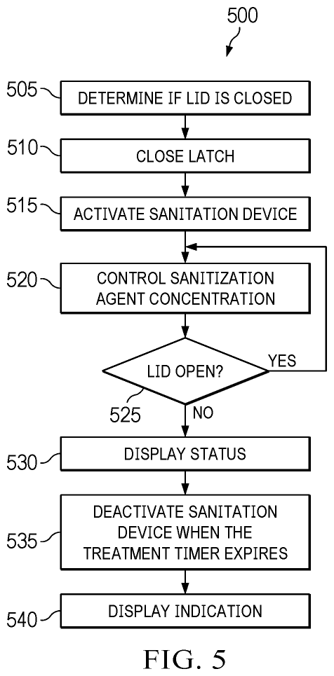

FIG. 5 depicts another example of a method of sanitization 500. At process 505, a controller (e.g., controller 205) associated with an enclosure (e.g., delivery receptacle 201) determines whether a lid or door (e.g., lid 202) of the enclosure is currently open or closed. Closing of the lid may be signaled to the controller by a sensor (e.g., a sensor monitoring the electronic lock 204) upon closing, the controller may send an inquiry to the sensor prompting a return signal indicating the current status, or the controller may maintain a status of a lock based on previously sent instructions to control the lock. The controller may also determine whether an article has been received in the compartment. In this regard, the controller may: 1) assume that an article has been placed in the compartment as a result of the lid having been closed by a user; 2) assume that an article has been placed in the compartment as a result of having received and/or authenticated a request for access from a driver device; and/or 3) a sensor may indicate to the controller that the presence of a package has been detected. The latter may help distinguish from a closing of the lid after removal of a package from the enclosure. Further, any other suitable techniques may be used to determine that an article has been placed in the compartment.

At process 510, an electronic lock (e.g., electronic lock 204) latches to secure the lid of the enclosure. The locking may be a required feature of the sanitization system or may be an option which is enabled by the user via a local or remote user interface. In some examples, the enclosure may allow temporary access to a delivery person to enable placement of additional articles in the enclosure. This may be permitted by receipt an appropriate security code via a local or remote user interface or by authentication processes executed between the controller and a remote access device (e.g., a package scanner or other delivery driver device).

At process 515, one or more sanitization devices (e.g., sanitization device 206), which may in some examples include an ozone generator, are activated. A sanitization device may be configured to dispense a sanitization agent (e.g., generate ozone) at a maximum output capacity, or the dispensing may be regulated, for example by a proportional-integral-derivative (PID) control loop, by a controller tasked with management of the treatment cycle (which may be the controller of the enclosure or a controller of the sanitization device), or by a flow regulator. The sanitization device may have a fan which can be activated to help circulate the sanitization agent throughout the compartment. Additional accessory fans in disposed in locations within the compartment may also be activated to aid in agent circulation. One or more sensors may monitor a concentration level of a sanitization agent within the compartment or monitor a dispensed volume of the sanitization agent as it flows from the sanitization device. The one or more sensors may send monitoring signals to the controller managing the treatment cycle. During process 515, the controller tasked with management of the treatment cycle initiates a treatment timer for executing a defined period of exposure of the article to the sanitization agent. The defined period may be determined based on a variety of factors including, but not limited to, a type of article received, a type of sanitization agent being used, a concentration of the sanitization agent, a type of pathogen expected, etc. For example, a 30-minute treatment period may be a known effective amount of time for an ozone concentration of approximately 3 ppm to effectively sanitize a cardboard box.

At process 520, one or more agent sensors (e.g., agent sensor 209) is used to provide input to the controller for monitoring the concentration of a sanitization agent which may permit the controller to modify a dispensing quantity of sanitization agent from the sanitization device to achieve and/or maintain a target concentration. A control algorithm executed by the controller may be a simple on-off control with one or more of a minimum or maximum threshold value or may be a more sophisticated PID, PI, or PD control of the sanitization device to modulate the dispensing quantity with avoidance of excessive or insufficient concentrations which may help reduce total treatment time.

At process 525, the controller determines whether the lid has been opened. If so, the method may return to process 515, restarting the treatment timer. This interruption of the method may occur, for example, if a subsequent delivery is made following the initiation of the treatment cycle. If it is determined that the lid has not been opened, the method continues to process 530. In some examples, if the method must return to process 515, the controller may determine a reduced treatment timer value is sufficient based on a period of exposure that is determined to have occurred before the interruption in the treatment cycle. This determination may be based in part on the concentration of sanitization agent previously dispensed, the period of exposure the article received prior to the lid being opened, the duration of the lid being open, and/or the concentration of sanitization agent remaining after closing of the lid. In some examples, the controller may determine that it is necessary to return to process 510 and dispense additional sanitization agent if the concentration was reduced by more than an acceptable amount by the lid being opened.

At process 530, a user interface of the display 214 indicates the status of the treatment cycle. For example, a certain color of light may be activated indicating a stage of the treatment cycle in process. A display which is part of user interface may indicate the treatment cycle is ongoing and may indicate a remaining time left in the cycle. Audio cues may also be provided by the user interface. The controller may also transmit a status of the treatment cycle to a remote interface, for example, via a wireless communication module.

At process 535, when the treatment timer expires, the sanitization device is deactivated. A timer associated with a waiting period may be initiated. The waiting period timer value may be user selected via the user interface or may be controlled by wireless connection to controller. The controller may also determine the waiting period timer value based on any number of factors including, but not limited to, sensor data, the volume of the compartment, a type of article delivered, a type of sanitization agent used, and/or the dispensing capacity of the sanitization device. In some examples of method 500, the waiting period timer may be based on the time it takes for a sanitization agent to naturally break down and/or escape the compartment and to achieve an innocuous concentration inside the compartment. In some examples, the controller monitors sanitization agent concentration using one or more agent sensors. The waiting period may be reduced if the controller determines that the concentration is reducing more quickly than estimated. The waiting period may be increased if the controller determines the concentration is reducing more slowly that estimated. Upon determining the concentration is below a predetermined threshold indicating an innocuous concentration, based on a default setting or user-modifiable setting, the controller may unlock the electronic lock or permit the lock to be unlocked by a user. Unlocking or permitted unlocking may occur upon expiration of the waiting period timer or sooner if the controller determines the concentration of sanitization agent has dropped below the predetermined threshold.

At process 540, the user interface of the enclosure may provide an indication to a user that the treatment cycle has finished and the lid may be opened for removal of the article. For example, a particular light may be activated or a message displayed on the display screen. A notification may also be sent to a user's mobile device or other remote interface.

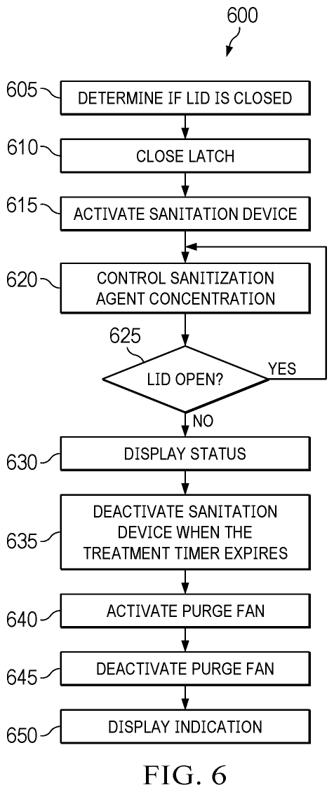

FIG. 6 depicts another example of a method of sanitization 600. At process 605, a controller (e.g., controller 205) associated with an enclosure (e.g., delivery receptacle 201) determines whether a lid or door (e.g., lid 202) of the enclosure is currently open or closed. Closing of the lid may be signaled to the controller by a sensor (e.g., a sensor monitoring the electronic lock 204) upon closing, the controller may send an inquiry to the sensor prompting a return signal indicating the current status, or the controller may maintain a status of a lock based on previously sent instructions to control the lock. The controller may also determine whether an article has been received in the compartment. In this regard, the controller may: 1) assume that an article has been placed in the compartment as a result of the lid having been closed by a user; 2) assume that an article has been placed in the compartment as a result of having received and/or authenticated a request for access from a driver device; and/or 3) a sensor may indicate to the controller that the presence of a package has been detected. The latter may help distinguish from a closing of the lid after removal of a package from the enclosure. Further, any other suitable techniques may be used to determine that an article has been placed in the compartment.

At process 610, an electronic lock (e.g., electronic lock 204) latches to secure the lid of the enclosure. The locking may be a required feature of the sanitization system or may be an option which is enabled by the user via a local or remote user interface. In some examples, the enclosure may allow temporary access to a delivery person to enable placement of additional articles in the enclosure. This may be permitted by receipt an appropriate security code via a local or remote user interface or by authentication processes executed between the controller and a remote access device (e.g., a package scanner or other delivery driver device). It should be appreciated that in some examples, a lock may be omitted and a sanitization system may be configured to operate only when the lid or door is closed or may be configured to operate regardless of lid position.

At process 615, one or more sanitization devices (e.g., sanitization device 206), which may in some examples include an ozone generator, are activated. A sanitization device may be configured to dispense a sanitization agent (e.g., generate ozone) at a maximum output capacity, or the dispensing may be regulated, for example by a proportional-integral-derivative (PID) control loop, by a controller tasked with management of the treatment cycle (which may be the controller of the enclosure or a controller of the sanitization device), or by a flow regulator. The sanitization device may have a fan which can be activated to help circulate the sanitization agent throughout the compartment. Additional accessory fans in disposed in locations within the compartment may also be activated to aid in agent circulation. One or more sensors may monitor a concentration level of a sanitization agent within the compartment or monitor a dispensed volume of the sanitization agent as it flows from the sanitization device. The one or more sensors may send monitoring signals to the controller managing the treatment cycle. During process 615, the controller tasked with management of the treatment cycle initiates a treatment timer for executing a defined period of exposure of the article to the sanitization agent. The defined period may be determined based on a variety of factors including, but not limited to, a type of article received, a type of sanitization agent being used, a concentration of the sanitization agent, a type of pathogen expected, etc. For example, a 30-minute treatment period may be a known effective amount of time for an ozone concentration of approximately 3 ppm to effectively sanitize a cardboard box.

At process 620, one or more agent sensors (e.g., agent sensor 209) is used to provide input to the controller for monitoring the concentration of a sanitization agent which may permit the controller to modify a dispensing quantity of sanitization agent from the sanitization device to achieve and/or maintain a target concentration. A control algorithm executed by the controller may be a simple on-off control with one or more of a minimum or maximum threshold value or may be a more sophisticated PID, PI, or PD control of the sanitization device to modulate the dispensing quantity with avoidance of excessive or insufficient concentrations which may help reduce total treatment time including purging.

At process 625, the controller determines whether the lid has been opened. If so, the method may return to process 615, restarting the treatment timer. This interruption of the method may occur, for example, if a subsequent delivery is made following the initiation of the treatment cycle. If it is determined that the lid has not been opened, the method continues to process 630. In some examples, if the method must return to process 615, the controller may initiate a second treatment timer, with the same timer value as the first treatment timer or a different timer value (e.g., a preset reduced value for use following an interruption), or may determine a reduced treatment timer value is sufficient based on a period of exposure that is determined to have occurred before the interruption in the treatment cycle. This determination may be based in part on the concentration of sanitization agent previously dispensed, the period of exposure the article received prior to the lid being opened, the duration of the lid being open, and/or the concentration of sanitization agent remaining after closing of the lid. In some examples, the controller may determine that it is necessary to return to process 610 and dispense additional sanitization agent if the concentration was reduced by more than an acceptable amount by the lid being opened.

At process 630, a user interface on the display 214 indicates the status of the treatment cycle. For example, a certain color of light may be activated indicating a stage of the treatment cycle in process. A display which is part of user interface may indicate the treatment cycle is ongoing and may indicate a remaining time left in the cycle. Audio cues may also be provided by the user interface. The controller may also transmit a status of the treatment cycle to a remote interface, for example, via a wireless communication module.

At process 635, when the treatment timer expires, the sanitization device is deactivated.

At process 640, active purging may be initiated to reduce the waiting period. Sanitization agent may be pushed out of the compartment by a purge fan through an opening leading either directly outside, for example at or near ground level, or through an exhaust vent (e.g., exhaust vent 211) to a desired location. In some examples, a catalyst module (not shown) may be disposed within the compartment. The catalyst module may be disposed along a path through which the sanitization agent exits the compartment. In some examples, the catalyst module may be disposed in-line within an exhaust vent or directly in front of or behind a purge fan. The catalyst module may be configured for replacement periodically or upon depletion of the catalyst through use. The catalyst module may render the sanitization agent less toxic or reduce its concentration in the exhaust stream. A catalyst module may be disposed within the compartment in a location away from the purge fan to reduce the concentration of the sanitization agent within the compartment. During active purging, in some examples, the controller monitors sanitization agent concentration using one or more agent sensors. Upon determining the concentration is below a predetermined threshold indicating an innocuous concentration, based on a default setting or user-modifiable setting, the controller may unlock the electronic lock or permit the lock to be unlocked by a user.

At process 645, the purge fan(s) is deactivated.

At process 650, the user interface of the enclosure may provide an indication to a user that the treatment cycle has finished and the lid may be opened for removal of the article. For example, a particular light may be activated or a message displayed on the display screen. A notification may also be sent to a user's mobile device or other remote interface.

The various processes described in relation to methods 300, 400, 500, and 600 may be performed in different combinations beyond what is expressly described for each example method. For example, a sanitization system may perform active purging, as described in relation to method 600, in the method 300. As another example, the method 600 may include a waiting timer such as in process 435 of method 400 in addition or instead of active purging. In some examples, a user may be able to configure the various parameters to exercise a degree of control over the processes of each method via a user interface. In some examples, each of the processes may be predefined in memory of the controller and may be restricted from modification by a user.

It should be appreciated that, in some examples, the enclosure may be used as a sanitization unit by a homeowner or user without a delivery. For example, a user may purchase articles at a store and, upon returning home, place the articles in the enclosure and initiate a treatment cycle. In some examples, a user may place food items, such as raw vegetables, into the enclosure to treat them prior to eating or cooking. In this manner, the enclosure may be utilized as a kitchen appliance.

It should be appreciated the described example methods herein may be modified as appropriate based on a type of enclosure. For example, any of the described methods may omit processes associated with locking and unlocking of a lid or door when the enclosure does not have a lid or door suitable for locking or when it would be impractical to lock the lid or door, such as in a postal mailbox for receiving parcels to be delivered (e.g., mailbox 110). Further, when a sanitization agent being used is believed to be innocuous to humans or pets at any concentration employed within the compartment, there may be no need for locking and unlocking. Further, some enclosure may not be fitted with certain described components, such as a purge fan and/or exhaust vent. Accordingly, it will be understood that the processes of the described examples associated with active purging may be omitted.

The sanitization systems and methods described herein may be used for various applications, as will be appreciated by those experienced in the art.

What is claimed is:

1. A method of sanitization, comprising:
determining a door of an enclosure is closed, the door separating a compartment within the enclosure from ambient air; and
performing a sanitization process, the sanitization process comprising:
determining an article has been placed into the compartment;
initiating a treatment timer;
introducing a sanitization agent into a volume of the compartment with a sanitization device; and
determining the treatment timer has expired.

2. The method of claim 1, further comprising:
determining the door of the enclosure has opened after initiating the treatment timer and before determining the treatment timer has expired; and
reinitiating the treatment timer in response to determining the door has opened.

3. The method of claim 1, further comprising:
providing a visual indication of a status of the sanitization process on a display.

4. The method of claim 1, further comprising:
initiating a depletion timer in response to determining the treatment timer has expired; and
providing an indication that the sanitization process is complete in response to expiration of the depletion timer.

5. The method of claim 1, further comprising:
initiating a purge timer in response to determining the treatment timer has expired;
activating a purge fan configured to purge air inside the compartment to the ambient air; and
deactivating the purge fan in response to determining the purge timer has expired.

6. The method of claim 1, further comprising:
monitoring a concentration of the sanitization agent within the compartment, wherein the treatment timer is initiated in response to the monitored concentration reaching a predetermined target concentration.

7. The method of claim 1, further comprising:
monitoring a concentration of the sanitization agent within the compartment; and
providing an indication that the sanitization process is complete in response to determining the monitored concentration is less than a predetermined threshold.

8. The method of claim 1, further comprising:
locking the door of the enclosure during the sanitization process.

9. The method of claim 8, further comprising:
unlocking the door of the enclosure after initiating the treatment timer and before determining the treatment timer has expired in response to authenticating a received request for access into the compartment.

10. A sanitization system, comprising:
an enclosure configured to receive and secure one or more articles, the enclosure having a door separating a compartment within the enclosure from ambient air;
a controller; and
a sanitization device;
wherein the controller is configured to:
determine an article has been placed into the compartment;
initiate a treatment timer;
instruct the sanitization device to introduce a sanitization agent into a volume of the compartment; and
determine the treatment timer has expired.

11. The sanitization system of claim 10, further comprising:
a locking mechanism configured to receive a command from the controller and actuate a lock in response to the received command, wherein the controller is further configured to transmit a command to the locking mechanism to actuate the lock to a locked configuration prior to introducing the sanitization agent into the volume.

12. The sanitization system of claim 11, wherein the controller is further configured to:
initiate a depletion timer in response to determining the treatment timer has expired; and
prevent unlocking of the locking mechanism until expiration of the depletion timer.

13. The sanitization system of claim 10, further comprising:
one or more sensors, wherein the controller is configured to monitor a concentration of the sanitization agent within the volume based on signals received from the one or more sensors.

14. The sanitization system of claim 13, further comprising:
one or more purge fans in operative communication with the controller, wherein the controller is further configured to:
activate the one or more purge fans;
monitor the concentration of the sanitization agent within the volume; and
deactivate the one or more purge fans in response to determining the concentration is below a threshold value.

15. The sanitization system of claim 13, wherein the controller is further configured to:
determine the monitored concentration of the sanitization agent within the volume is less than a target concentration; and
instruct the sanitization device to dispense an additional quantity of sanitization agent in response to determining the monitored concentration is less than the target concentration.

16. The sanitization system of claim 13, wherein the controller is further configured to:
delay the initiation of the treatment timer or modify a timer value of the treatment value based on the monitored concentration of the sanitization agent within the volume.

17. A parcel receptacle, comprising:
an enclosure configured to receive and secure one or more parcels, the enclosure having a door separating a compartment within the enclosure from ambient air;
a sanitization device configured to introduce a sanitization agent into a volume of the compartment; and
a controller configured to:
communicate with a remote access device to selectively provide access into the compartment to delivery personnel;
determine a parcel has been placed into the compartment;
initiate a treatment timer;
instruct the sanitization device to introduce the sanitization agent into the volume of the compartment;
determine the treatment timer has expired; and
provide a visual indication that the treatment timer has expired.

18. The system of claim 17, wherein the sanitization device is an ozone generator and the sanitization agent is ozone.

19. The system of claim 18, wherein the controller is further configured to:
initiate a depletion timer in response to expiration of the treatment timer; and
prevent access into the compartment until expiration of the depletion timer, wherein a timer value of the depletion timer is based on a period of time associated with degradation of ozone.

20. The system of claim 17, further comprising:
one or more ozone sensors, wherein the controller is configured to monitor a concentration of ozone within the volume based on signals received from the one or more sensors; and
one or more purge fans in operative communication with the controller, wherein the controller is further configured to:
activate the one or more purge fans;
monitor the concentration of ozone within the volume during operation of the one or more purge fans; and
deactivate the one or more purge fans in response to determining the monitored concentration is below a threshold value.

21. A method of sanitization, comprising:
performing a sanitization process upon closure of a door separating a package-receiving compartment within a package-receiving enclosure from ambient air, the sanitization process comprising:
determining an article has been placed into the compartment;
initiating a treatment timer;
introducing a sanitization agent into a volume of the compartment with a sanitization device; and
determining the treatment timer has expired.

22. The method of claim 21, further comprising:
determining the door of the enclosure has opened after initiating the treatment timer and before determining the treatment timer has expired; and
reinitiating the treatment timer in response to determining the door has opened.
